(19) United States
(12) Patent Application Publication
(10) Pub. No.: US 2018/0085627 A1
(43) Pub. Date: Mar. 29, 2018

(54) ELECTRONIC DEVICE, CONSIDERATION SETTING METHOD AND NON-TRANSITORY COMPUTER-READABLE MEDIUM

(71) Applicant: CASIO COMPUTER CO., LTD., Tokyo (JP)

(72) Inventor: Kazunori KITA, Tokyo (JP)

(73) Assignee: CASIO COMPUTER CO., LTD., Tokyo (JP)

(21) Appl. No.: 15/676,339

(22) Filed: Aug. 14, 2017

(30) Foreign Application Priority Data
Sep. 23, 2016 (JP) 2016-186165

(51) Int. Cl. A63B 24/00; G06F 3/041; G06F 3/044; A63B 71/06; G09B 5/02; A61B 5/00

(52) U.S. Cl. CPC A63B 24/0062; G06Q 30/0279; G06F 3/044; A63B 71/0622; G09B 5/02; A61B 5/681; A63B 2220/12; A63B 2225/50; A63B 2220/836; A63B 2071/0663; A63B 2230/06; A63B 2230/30; A63B 2220/44; A63B 2220/56; A63B 2220/89; A63B 2024/0071; G06F 3/0412

(57) ABSTRACT

A wrist terminal includes a sensor information acquisition unit, a work/learning information acquisition unit, and a consideration setting unit. The sensor information acquisition unit acquires user exercise information and/or user biological information. The work/learning information acquisition unit acquires work/learning information on a user based on the user exercise information and/or the user biological information. The consideration setting unit sets information representing a consideration for work/learning information on a user based on the work/learning information on the user, which is acquired by the work/learning information acquisition unit.

### CROSS-REFERENCE TO RELATED APPLICATIONS

This application is based upon and claims the benefit of priority under 35 USC 119 of Japanese Patent Application No. 2016-186165 filed on Sep. 23, 2016 the entire disclosure of which, including the description, claims, drawings, and abstract, is incorporated herein by reference in its entirety.

### BACKGROUND OF THE INVENTION

### 1. Field of the Invention

The present invention relates to an electronic device and consideration setting method and non-transitory computer-readable medium.

### 2. Description of the Related Art

Usually, with respect to an action such as a labor or learning, money is paid for the labor, or a learning progress certification is issued. In this case, to certify achievement of the labor and learning, a worker or a person who is learning self-manages the achievement, or a causer manages the achievement. However, such a management method is generally associated with complications.

Here, JP 2000-245713 A discloses a technique to analyze user's action by detecting user's motion.

In addition, JP 2016-043260 A discloses a technique to analyze the amount of work and energy consumption of user's motion by detecting the motion.

### BRIEF SUMMARY OF THE INVENTION

According to an aspect of the present invention, there is provided an electronic device including a processor configured to

acquire user exercise information and/or user biological information,

acquire work information representing a work regarding a user or learning information representing learning regarding the user based on the user exercise information and/or the user biological information, and

set information representing a consideration based on the work information or the learning information.

According to another aspect of the present invention, there is provided a consideration setting method performed by an electronic device including a processor, configured to acquire user exercise information and/or user biological information, the consideration setting method including the steps of:

acquiring work information representing a work regarding a user or learning information representing learning regarding the user based on the user exercise information and/or the user biological information; and

setting information representing a consideration based on the acquired work information or the learning information.

According to another aspect of the present invention, there is provided a non-transitory computer-readable medium including a program stored thereon, which, when executed on a processor, realizes:

an exercise/biological information acquisition function configured to acquire user exercise information and/or user biological information;

a work/learning information acquisition function configured to acquire work information representing a work regarding a user or learning information representing learning regarding the user based on the user exercise information and/or the user biological information; and

a consideration setting function configured to set information representing a consideration based on the work information or the learning information acquired by the work/learning information acquisition function.

### BRIEF DESCRIPTION OF THE SEVERAL VIEWS OF THE DRAWING

FIG. 1 illustrates configuration of a wrist terminal as an embodiment of an electronic device according to the present invention, FIG. 1A illustrates an external appearance configuration, and FIG. 1B is a block diagram indicating a hardware configuration;

FIG. 2 is a functional block diagram indicating a functional configuration to perform consideration setting processing in the functional configuration of the wrist terminal illustrated in FIG. 1;

FIG. 3 is a schematic diagram indicating an example of an action determination condition table;

FIG. 4 is a schematic diagram indicating an example of a consideration setting condition table;

FIG. 5 is a flowchart describing a flow of consideration setting processing performed by the wrist terminal illustrated in FIG. 1 including the functional configuration indicated in FIG. 2;

FIG. 6 is a schematic diagram indicating a system configuration of a consideration setting system according to a second embodiment of the present invention;

FIG. 7 is a block diagram indicating a hardware configuration of a consideration setting server as an embodiment of an electronic device according to the present invention;

FIG. 8 is a functional block diagram indicating a functional configuration to perform terminal-side consideration setting processing in a functional configuration of a wrist terminal according to the second embodiment;

FIG. 9 is a functional block diagram indicating a functional configuration to perform server-side consideration setting processing in a functional configuration of the consideration setting server indicated in FIG. 7;

FIG. 10 is a flowchart describing a flow of a terminal-side consideration setting processing performed by a wrist terminal according to the second embodiment; and

FIG. 11 is a flowchart describing a flow of the server-side consideration setting processing performed by the consideration setting server.

### DETAILED DESCRIPTION OF THE INVENTION

Embodiments of the present invention will be explained with reference to the drawings.

Embodiments of the present invention will be described below with reference to the accompanying drawings.

### First Embodiment

An electronic device according to a first embodiment acquires user exercise information and user biological information by a sensor and grants information representing a predetermined consideration in the case where it is determined that the user's action represented by the acquired exercise information or biological information is an action for which a consideration should be given. (Meaning of a consideration includes meaning of a reward.) At this time, whether user's action is an action for which a consideration should be given is determined based on preset action determination conditions. In the case where user's action is determined to be an action for which a consideration should be given, information representing a predetermined consideration is set based on conditions for setting a consideration to be given to the action. In the embodiment, when the information representing a consideration is set, information representing user's action is appropriately authenticated. The information representing the set consideration can be accumulated in response to user's request and can be used by a user as a point.

Therefore, since a consideration for user's action is generated in accordance with a predetermined standard, the user can work and volunteer with high action motivation and high fairness and satisfaction.

As described above, in the electronic device according to the embodiment, it is possible to appropriately add value for user's action and further simply manage user's action.

Specific embodiments will be described below.

Hardware Configuration

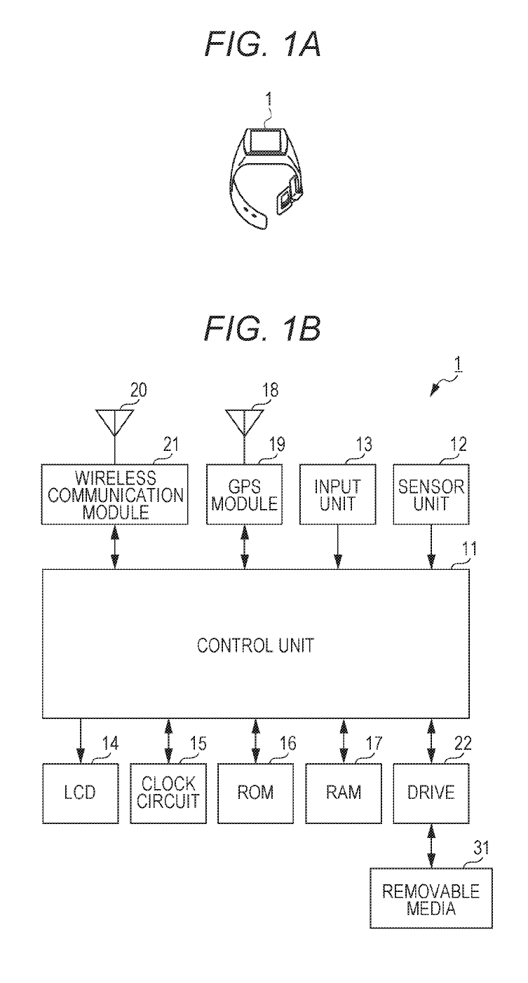

FIG. 1 illustrates a configuration of a wrist terminal 1 as an embodiment of the electronic device according to the present invention. FIG. 1A illustrates an external appearance configuration. FIG. 1B is a block diagram indicating a hardware configuration.

As illustrated in FIG. 1A, the wrist terminal 1 is a wrist watch-type electronic device having smartphone-like functions.

Further, as illustrated in FIG. 1B, the wrist terminal 1 includes a control unit 11, a sensor unit 12, an input unit 13, a liquid crystal display (LCD) 14, a clock circuit 15, a read only memory (ROM) 16, a read access memory (RAM) 17, a GPS antenna 18, a GPS module 19, a wireless communication antenna 20, a wireless communication module 21, and a drive 22.

The control unit 11 includes an arithmetic processing unit such as a central processing unit (CPU) and controls a whole operation of the wrist terminal 1. For example, the control unit 11 performs various processing in accordance with a program stored in the ROM 16, such as a program for consideration setting processing (to be described later).

The sensor unit 12 includes various sensors such as a biological sensor (such as a pulse sensor and a blood pressure sensor), a geomagnetic sensor, a three-axis acceleration sensor, a pressure sensor, and a temperature sensor.

The input unit 13 includes various buttons and a capacitance-type or resistance film-type position input sensor laminated in a display region of the LCD 14, and various types of information is input in response to an instruction operation by a user.

The LCD 14 outputs an image in accordance with an instruction of the control unit 11. For example, the LCD 14 displays various images and a user interface screen. In the embodiment, the position input sensor of the input unit 13 is superimposed on the LCD 14, and a touch panel is formed.

The clock circuit 15 generates a time signal from a signal generated by a system clock or an oscillator and outputs the present time.

The ROM 16 stores information on such as a control program to be performed by the control unit 11. For example, the ROM 16 stores a program for consideration setting processing to be described later. In the embodiment, a program for this consideration setting processing is provided by a main entity having a formal right for setting a consideration. Therefore, information representing a consideration set by performing a program for consideration setting processing can be treated with authenticity.

The RAM 17 provides a work area for performing each processing by the control unit 11.

The GPS antenna 18 converts radio waves issued from a GPS satellite into electric signals and outputs the converted electrical signals (hereinafter called, “GPS signal”) to the GPS module 19.

The GPS module 19 detects a position of the wrist terminal 1 (latitude, longitude, and altitude) and the present time indicated by a GPS, based on the GPS signal input from the GPS antenna 18. The GPS module 19 outputs information on the detected position and present time to the control unit 11.

The wireless communication antenna 20 is an antenna which can receive radio waves with a frequency corresponding to wireless communication used by the wireless communication module 21 and, for example, includes a loop antenna and a rod antenna. The wireless communication antenna 20 sends a wireless communication electric signal input from the wireless communication module 21 as electromagnetic waves. Further the wireless communication antenna 20 converts received electromagnetic waves into an electric signal, and outputs the electric signal to the wireless communication module 21.

The wireless communication module 21 sends a signal to other devices via the wireless communication antenna 20 in accordance with an instruction from the control unit 11. Further, the wireless communication module 21 receives a signal sent from other devices and outputs information indicated by the received signal to the control unit 11. In the embodiment, the wireless communication module 21 performs communication based on the communication standard for Bluetooth low energy/Bluetooth LE (trademark) (hereinafter called “BLE”) and the communication standard for wireless fidelity (Wi-Fi). Further, the wireless communication module 21 can communicate with other wrist terminals 1 by a beacon of the BLE.

The drive 22 appropriately includes a removable media 31 including such as a magnetic disc, an optical disk, a magneto-optical disk, or a semiconductor memory. The removable media 31 can store various data such as image data.

Functional Configuration

Next, among functional configurations of the wrist terminal 1, a functional configuration to perform consideration setting processing will be described.

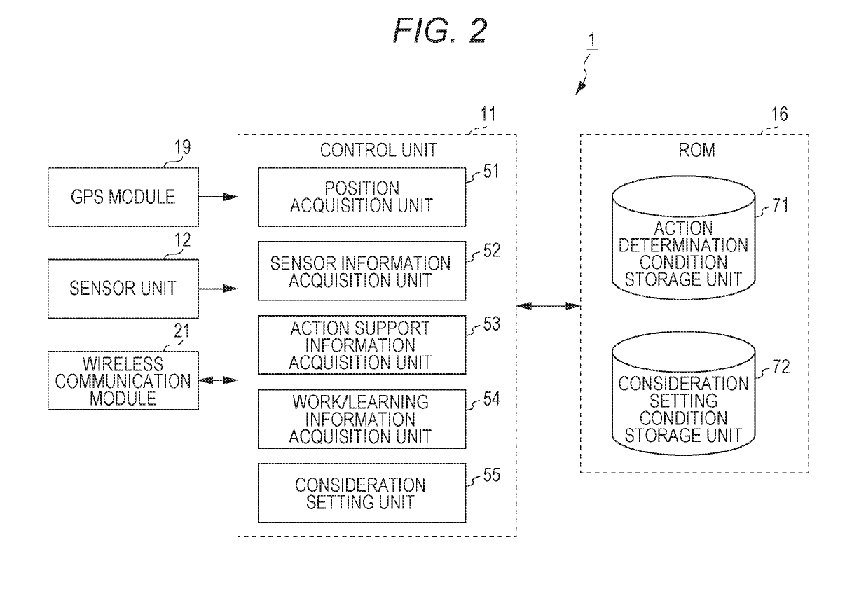

FIG. 2 is a functional block diagram indicating a functional configuration to perform consideration setting processing among functional configurations in the wrist terminal 1 in FIG. 1.

The consideration setting processing is a series of processing to acquire user exercise information and user biological information by a sensor and set information representing a consideration for user's action based on the acquired exercise information or biological information.

In the case where the consideration setting processing is performed, as illustrated in FIG. 2, a position acquisition unit 51, a sensor information acquisition unit 52, an action support information acquisition unit 53, a work/learning information acquisition unit 54, and a consideration setting unit 55 function in the control unit 11. Further, an action determination condition storage unit 71 and a consideration setting condition storage unit 72 are provided in a region of the ROM 16.

The action determination condition storage unit 71 stores an action determination condition table corresponding to various action patterns represented by user exercise information and user biological information and information representing a work (hereinafter called “work information) or information representing learning (hereinafter called “learning information”), estimated from the action pattern. The action pattern is an action unit in which exercise information or biological information in a predetermined time period is compiled. Further, the work includes user's action to accomplish a specific object, and the learning includes user's action to acquire knowledge and a skill. Hereinafter, the work information and learning information are collectively and appropriately called “work/learning information”.

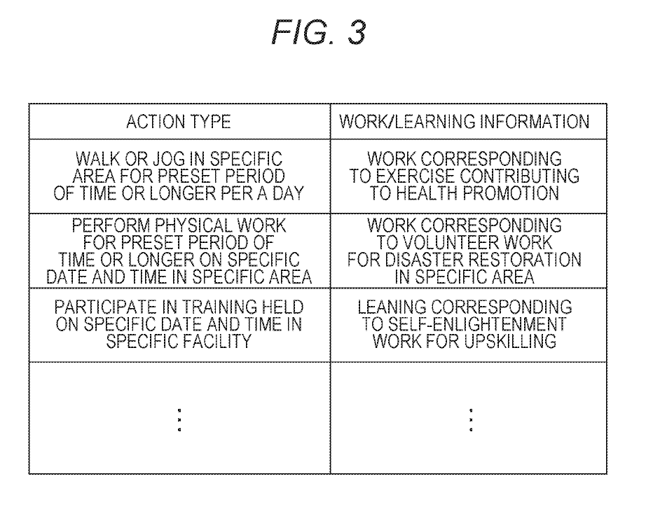

FIG. 3 is a schematic diagram indicating an example of an action determination condition table.

As illustrated in FIG. 3, in the action determination condition table, “action kinds” representing various action patterns represented by user exercise information and user biological information and “work/learning information” corresponding to the action kinds are stored association with each other.

For example, on the first line of the action determination condition table, an action kind, “walking or jogging in a specific area for preset period of time or longer per a day”, is defined, and as work information corresponding to this action kind, “a work corresponding to exercise contributing to health promotion” is defined. This is, for example, performed as a business to encourage exercise for health promotion by such as a community association, and a consideration for the work information can be paid by a budget for health promotion measures.

Further, on the second line of the action determination condition table, an action kind, “performing a physical work for preset period of time or longer on specific date and time in a specific area”, is defined, and as work information corresponding to this action kind, “a work corresponding to a volunteer work for disaster restoration in a specific area” is defined. This is, for example, performed as a volunteer business sponsored by a public entity for disaster restoration. A consideration for the work information can be paid by a subsidy provided to the public entity from a relevant government office.

Further, on the third line of the action determination condition table, an action kind, “participating in a training held on a specific date and time at a specific facility”, is defined, and as learning information corresponding to this action kind, “learning corresponding to a self-enlightenment work for upskilling” is defined. This is, for example, performed as a support business for upskilling of workers by a company or a relevant government office, and a consideration for the learning information can be paid by such as a subsidy from the company to which a user belongs or the relevant government office.

As described above, in the action determination condition table, conditions to determine various action patterns as the work/learning information are defined. These action patterns include a physical action which is mainly an action by using user's body and a mental action which is mainly an action by using user's brain. Each of the physical action and the mental action, combination of the physical action and the mental action, combination of a plurality of the physical actions, and combination of a plurality of mental actions are defined.

With reference to FIG. 2 again, the consideration setting condition storage unit 72 stores a consideration setting condition table associated with “a consideration granting condition”, “a consideration granted amount”, “a consideration granting entity”, and “an authorization entity” (hereinafter called “consideration setting conditions”) regarding each work/learning information defined in the action determination condition table.

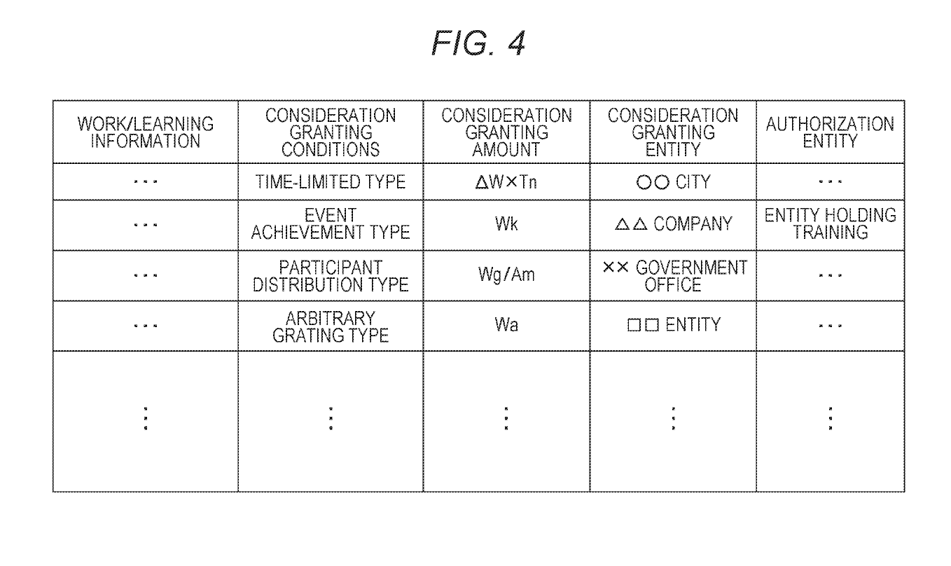

FIG. 4 is a schematic diagram indicating an example of the consideration setting condition table.

As illustrated in FIG. 4, each piece of work/learning information defined in the action determination condition table and “a consideration granting condition”, “a consideration granted amount”, “a consideration granting entity”, and “an authorization entity” are mutually associated and stored in the consideration setting condition table.

“A consideration granting condition” indicates kinds of consideration granting forms with respect to the work/learning information and defines “a time-limited type” in which a consideration generates in accordance with work hours, “an event achievement type” in which a consideration generates when a preset event is achieved, “a participant distribution type” in which the total of a consideration is distributed to participants engaged in a work in which the total of a consideration is set in advance, and “an arbitrary grating type” in which a consideration is arbitrarily granted by a provider of the work.

“A consideration granted amount” represents the granted amount of specific consideration for the work/learning information. For example, in the case of the time-limited type consideration, as the amount of granted considerations, “a time unit ΔW×a work time Tn” is defined. Further, in the case of the event achievement-type consideration, as the amount of granted considerations, “a preset consideration for a work Wk” is defined. Further, in the case of the participant distribution-type consideration, as the amount of granted considerations, “a total amount of preset considerations for a work Wg/the number of persons engaged in the work Am” is defined. Further, in the case of the arbitrary granting-type consideration, as the amount of granted considerations, “a consideration arbitrarily granted by a provider of a work Wa” is defined.

“A consideration granting entity” represents an entity by which a consideration for the work/learning information is granted. The consideration granting entity is determined for each work/learning information. For example, as a consideration granting entity, in the consideration setting condition table, “◯◯ city” (a community association) is defined on the first line, “ΔΔ company” (a company) is defined on the second line, and “XX government office” (a government office) is defined on the third line, and “□□ entity” (a public entity) is defined on the fourth line.

“An authorization entity” is set in the case where the work/learning information needs to be authorized for granting a consideration, and the authorization entity is an entity which authorizes the work/learning information. For example, in the case where the work/learning information is “a work corresponding to a self-enlightenment activity for upskilling in a job”, an action kind, “participating in a training held on a specific date and time at a specific facility”, detected in the wrist terminal 1 is authorized by an entity holding a training based on such as an attendance situation at a training site. In this case, identification information representing a user of the wrist terminal 1 (user ID) and data of the action kind detected in the wrist terminal 1 are sent to a server operated by a training holding entity, and an authentication result in the server can be sent to an entity for granting a consideration for the work/learning information, and an authentication result with an electronic signature can be sent to the wrist terminal 1.

With reference to FIG. 2 again, the position acquisition unit 51 acquires a positioning position of the wrist terminal 1.

Specifically, the position acquisition unit 51 acquires a positioning position of the wrist terminal 1 based on position information acquired from the GPS module 19. The position acquisition unit 51 accumulates and stores the positioning position of the wrist terminal 1 in the ROM 16.

The sensor information acquisition unit 52 acquires output results of various sensors included in the sensor unit 12. For example, the sensor information acquisition unit 52 acquires user biological information of the wrist terminal 1 output by a biological sensor and user exercise information of the wrist terminal 1 output by a three-axis acceleration sensor. The sensor information acquisition unit 52 accumulates and stores the acquired biological information and exercise information in the ROM 16.

The action support information acquisition unit 53 acquires support information (hereinafter called “action support information”) to determine an action of a user of the wrist terminal 1 based on information output by the position acquisition unit 51 and the sensor information acquisition unit 52 and various user information stored in the wrist terminal 1. For example, the action support information acquisition unit 53 acquires information that a user participates in a training and a volunteer work, with reference to user's schedule data. By using this action support information, it can be easily determined what training a user of the wrist terminal 1 is participating in or whether the user is participating in a volunteer work. In the case where the action support information acquisition unit 53 does not acquire action support information, user's action is determined by appropriately inputting necessary information for the user.

The work/learning information acquisition unit 54 determines whether user's action pattern is an action pattern associated with the work/learning information (in other words, acquires the work/learning information for user's action) with reference to an action determination condition table based on position information of the wrist terminal 1 acquired by the position acquisition unit 51, user biological information and user exercise information acquired by the sensor information acquisition unit 52, and action support information acquired by the action support information acquisition unit 53.

For example, the work/learning information acquisition unit 54 determines that an action pattern of a user of the wrist terminal 1 matches an action information determination condition that “a physical work is performed for preset period of time or longer on specific date and time in a specific area” in the case where a positioning position acquired by the position acquisition unit 51 on specific date and time is in a specific area, and a time period of a physical work in the biological information and exercise information acquired by the sensor information acquisition unit 52 is determined to be equal to or longer than a set time period. Specifically, as work/learning information with respect to an action of a user of the wrist terminal 1, “a work corresponding to a volunteer work for disaster restoration in a specific are” is obtained. Whether the biological information and exercise information acquired by the sensor information acquisition unit 52 represents a physical work can be determined based on data whether a value indicating the biological information (such as a pulse and a blood pressure) and a value indicating the exercise information (such as an acceleration) are equal to or greater than a preset threshold time period and a preset threshold.

Further, for example, the work/learning information acquisition unit 54 determines that an action pattern of a user of the wrist terminal 1 matches an action information determination condition that “a user participates in a training held on a specific date and time at a specific facility” in the case where a positioning position acquired by the position acquisition unit 51 on a specific date and time corresponds to a position of a specific facility, and it is determined that action support information indicates that the user participates in a specific training. In other words, as the work/learning information on an action of a user of the wrist terminal 1, “learning corresponding to a self-enlightenment work for upskilling” is acquired. At this time, biological information and exercise information acquired by the sensor information acquisition unit 52 may also determine such as whether a user sits down and takes notes (whether a mental work is performed).

The work/learning information acquisition unit 54 notifies a user, for example, by displaying on the LCD 14 of the wrist terminal 1 that the work/learning information is not acquired, in the case where the work/learning information is not acquired.

Then, the work/learning information acquisition unit 54 accepts input of the work/learning information by a user. The work/learning information acquisition unit 54 adds a flag indicating the work/learning information by manual input to the work/learning information received herein.

With reference to a consideration setting condition table, the consideration setting unit 55 acquires “a consideration granting condition”, “a consideration granted amount”, “a consideration granting entity”, and “an authorization entity” (consideration setting conditions) based on the work/learning information determined that a user of the wrist terminal 1 has performed.

Then, the consideration setting unit 55 calculates the amount of granted consideration for the work/learning information determined that a user of the wrist terminal 1 is performed and specifies a an entity granting a consideration for an action of a user of the wrist terminal 1 based on position information of the wrist terminal 1 acquired by the position acquisition unit 51, user biological information and user exercise information acquired by the sensor information acquisition unit 52, and action support information acquired by the action support information acquisition unit 53. At this time, in the case where “an authorization entity” is set to the determined work/learning information, the consideration setting unit 55 requests authorization of the work/learning information to the authorization entity. In the case where the work/learning information is authorized by the authorization entity, the consideration setting unit 55 grants a consideration to a user of the wrist terminal 1. In this manner, the consideration setting unit 55 sets information representing a consideration for the work/learning information.

Operation

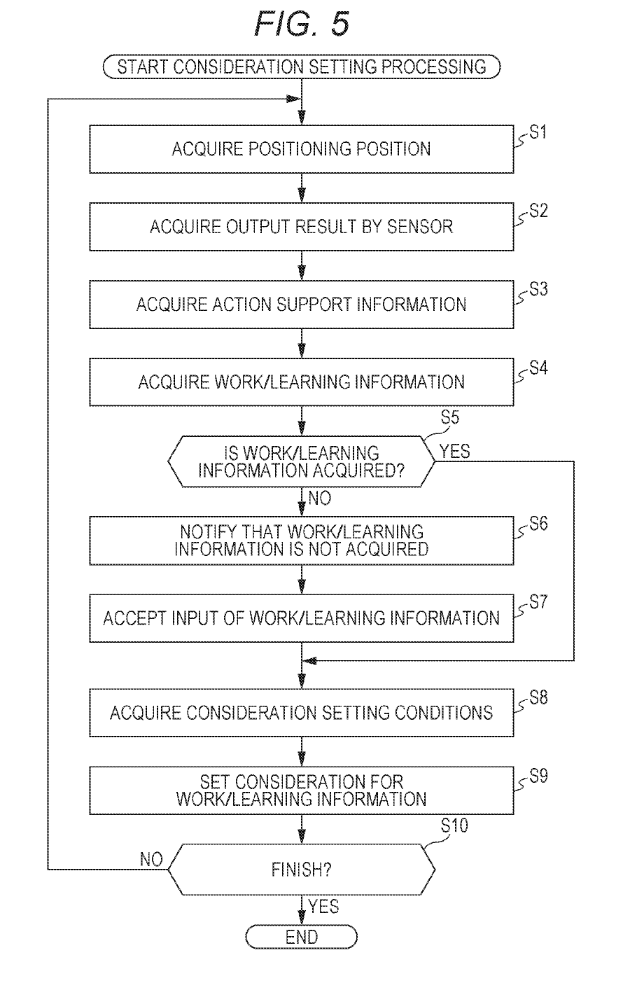

FIG. 5 is a flowchart describing a flow of consideration setting processing performed by the wrist terminal 1 indicated in FIG. 1 including the functional configuration indicated in FIG. 2.

The consideration setting processing is started when a user inputs an operation to start consideration setting processing to the input unit 13.

In step S1, the position acquisition unit 51 acquires a positioning position of the wrist terminal 1 based on position information acquired from the GPS module 19.

In step S2, the sensor information acquisition unit 52 acquires an output result of each sensor included in the sensor unit 12.

In step S3, the action support information acquisition unit 53 acquires action support information of a user of the wrist terminal 1 based on information output by the position acquisition unit 51 and the sensor information acquisition unit 52 and various user information stored in the wrist terminal 1.

In step S4, the work/learning information acquisition unit 54 acquires the work/learning information on user's action with reference to an action determination condition table based on position information of the wrist terminal 1 acquired by the position acquisition unit 51, user biological information and user exercise information, acquired by the sensor information acquisition unit 52, and action support information acquired by the action support information acquisition unit 53.

In step S5, the work/learning information acquisition unit 54 determines whether work/learning information is acquired.

In the case where the work/learning information is not acquired, it is determined NO in step S5, and step S6 is performed.

On the other hand, in the case where the work/learning information is acquired, it is determined YES in step S5, and step S8 is performed.

In step S6, the work/learning information acquisition unit 54 notifies a user, for example, by displaying on the LCD 14 of the wrist terminal 1 that the work/learning information is not acquired.

In step S7, the work/learning information acquisition unit 54 accepts input of the work/learning information by a user. In association with processing in step S7, a user manually inputs the work/learning information from a touch panel of the wrist terminal 1. The work/learning information acquisition unit 54 adds a flag indicating the work/learning information by manual input to the work/learning information received herein.

In step S8, the consideration setting unit 55 acquires a consideration setting condition based on the work/learning information determined that a user of the wrist terminal 1 has performed, with reference to a consideration setting condition table.

In step S9, the consideration setting unit 55 sets information representing a consideration for work/learning information. In the case where a flag indicating manually input work/learning information is added to the work/learning information, a consideration for the work/learning information may be held while it is confirmed whether the work/learning information is normally performed. In this case, at a timing of step S9, a user may be notified that authenticity of the work/learning information is being confirmed. The authenticity of the work/learning information may be confirmed by directly contacting between a user and an authorization entity or a consideration granting entity.

In step S10, the consideration setting unit 55 determines whether finish of consideration setting processing is instructed.

In the case where finish of the consideration setting processing is not instructed, it is determined NO in step S10, and step S1 is performed.

On the other hand, in the case where finish of the consideration setting processing is instructed, it is determined YES in step S10, and the consideration setting processing is finished.

### Specific Application Example

Promotion of Participation in Volunteer Activity

As an action to grant a consideration by the wrist terminal 1 according to the embodiment, a volunteer work, nursing and caring, a working service, a community activity, and a disaster support activity are targeted.

A user can freely participate in free and nonfree volunteers, like collecting points on a point card and stamps on a stamp card.

A user can acquire a consideration instead of a currency of a predetermined value when the user participates in a working service, a volunteer work, and a nursing support, a community event, and a local community activity, and a community contribution activity. As a consideration in the embodiment, in addition to a consideration instead of a currency of a predetermined value, a currency of a predetermined value itself may be acquired. This consideration can be used, for example, as a community currency, a volunteer currency, an electronic cash, a point on a point card, or a currency in a predetermined country. The consideration contributes to mutually providing a work and a duty and can be used for purchase of a product or a service. Therefore, the consideration can support participation in and activation of a working service, a volunteer work, and a community contribution activity.

In addition, fine work and labor such as minor work and help also can be converted into such as a point and a community currency, and therefore user's satisfaction can be increased.

Value of Consideration as Own Right

A consideration granted in the embodiment can be also used as a right that a user can receive nursing/caring, a working service, a work, and a duty in the future.

Therefore, in addition to a duty servicing volunteer spirit, conscience satisfaction, and a sense of mission, activities and labors performed when a user is healthy are accumulated as a point (consideration), and the point can be used for an insurance investment and a risk avoidance as a help for receiving a support, for example, when a user is aged and lives along.

In this case, limitations may be added that the point (consideration) acquired by a labor and an activity can be used by a user and a predetermined family and cannot be used outside the community.

Utilization as Certifying Units

Information representing a consideration set in the wrist terminal 1 according to the embodiment can be used as a certification to certify that a user participates in, attends, and finishes, or passes predetermined schools and classed, learning courses, training courses, credits acquired in learning and training, predetermined trainings (such as a fire drill), and various examinations. Specifically, in the case where user's action pattern is determined to correspond to predetermined learning information, information representing a consideration set for the learning information can be used as a certification of the predetermined learning information.

In this case, information representing a consideration is registered (issued) as a point of a learning credit, a point of skill acquisition, a certificate for receiving a course, a completion certificate, a passing certificate, a license, and a stamp on a stamp card. A user of the wrist terminal 1 acquires data on the certificate and records the point and the certificate for receiving a course in the form not being forged or reproduced.

As a result, the information representing a consideration in the embodiment can be used as a predetermined license certificate.

For example, the information representing a consideration in the embodiment can be used as a right certificate to engage in a work, a labor, a part-time job, an operation, and a labor provision, which require predetermined knowledge and skill.

Further, the information representing a consideration in the embodiment can be used as a proof of a predetermined right to enter, access, or pass through predetermined facilities, sites and gates such as schools, class rooms, classes, courses, training facilities, and test sites.

Utilization as Self-Enlightenment Tool

When a consideration for user's action is granted by the wrist terminal 1 according to the embodiment, a user can have a sense of accomplishment with respect to fine efforts when taking a course, studying, and taking a correspondence course.

Specifically, information representing a consideration for user's fine effort can be stored in visible forms such as a certificate, an electronic stamp, and a license, and therefore a user can be satisfied in the effort.

By the information representing a consideration accumulated in this manner, a user obtains benefits such that the user can have a right to take a higher-level course and a right to receive an education in other fields, and further the user can exchange the consideration into something that the user wants, receive a service, and objectively certify user's academic ability and skill.

Some of application targets have been exemplified above. The wrist terminal 1 according to the embodiment acquires user's exercise information and biological information by a sensor. In the case where it is determined that user's action pattern is an action pattern associated with the work/learning information, information representing a consideration is set for the work/learning information associated with the action pattern.

Therefore, since a consideration for user's action is generated in accordance with a predetermined standard, the user can work and volunteer with high action motivation and high fairness and satisfaction.

Therefore, according to the wrist terminal 1, a value can be further properly added to user's action.

### Second Embodiment

Next, a second embodiment of the present invention will be described.

In the embodiment, main processing performed in the work/learning information acquisition unit 54 and the consideration setting unit 55 of the wrist terminal 1 in the first embodiment is performed by a predetermined server in an embodiment of the electronic device according to the present invention.

System Configuration

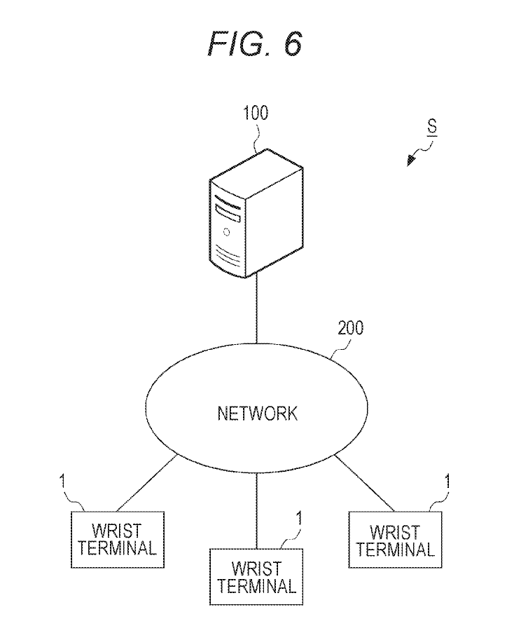

FIG. 6 is a schematic diagram indicating a system configuration of a consideration setting system S according to the second embodiment of the present invention.

As illustrated in FIG. 6, the consideration setting system S includes a wrist terminal 1 and a consideration setting server 100, and the wrist terminal 1 can communicate with the consideration setting server 100 via a network 200.

Hardware Configuration

In FIG. 6, a hardware configuration of the wrist terminal 1 is similar to configurations illustrated in FIGS. 1A and 1B in the first embodiment.

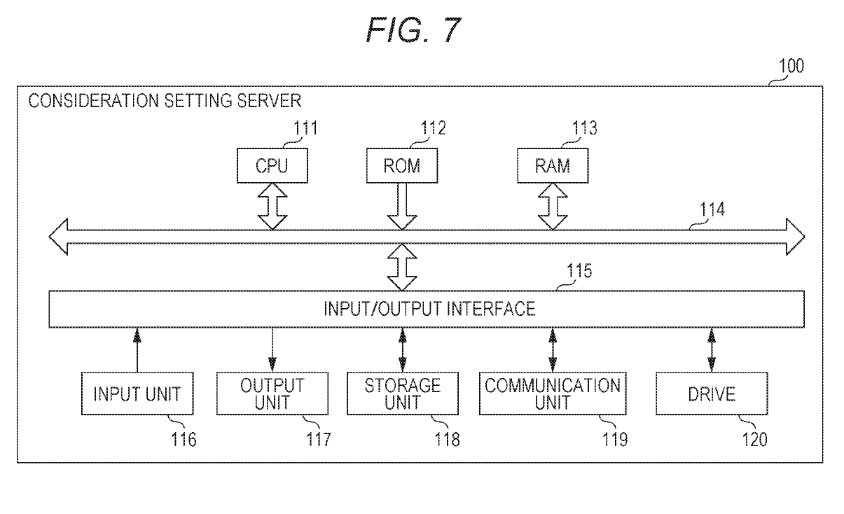

FIG. 7 is a block diagram indicating a hardware configuration of the consideration setting server 100.

As illustrated in FIG. 7, the consideration setting server 100 includes a CPU 111, a ROM 112, a RAM 113, a bus 114, an input/output interface 115, an input unit 116, an output unit 117, a storage unit 118, a communication unit 119, and a drive 120.

The CPU 111 executes a program recorded in the ROM 112 or various processing in accordance with a program loaded from the storage unit 118 to the RAM 113.

The RAM 113 also appropriately stores necessary data needed by the CPU 111 to perform each processing.

The CPU 111, the ROM 112, and the RAM 113 are mutually connected via the bus 114. This bus 114 is connected to the input/output interface 115. The input/output interface 115 is connected to the input unit 116, the output unit 117, the storage unit 118, the communication unit 119, and the drive 120.

The input unit 116 includes various buttons and inputs various information in response to an instruction operation by a user.

The output unit 117 includes such as a display and a speaker and outputs an image and a voice.

The storage unit 118 includes such as a hard disk or a dynamic random access memory (DRAM) and stores various data.

The communication unit 119 controls wireless or wired communication performed with another device via a network including the internet.

The drive 120 appropriately includes a removable media (not illustrated) including a magnetic disc, an optical disk, a magneto-optical disk, or a semiconductor memory. A program read from the removable media by the drive 120 is installed in the storage unit 118 as needed. Further, similar to the storage unit 118, the removable media stores various data stored in the storage unit 118.

Functional Configuration

Next, a functional configuration of the consideration setting system S will be described.

First, a functional configuration of the wrist terminal 1 will be described.

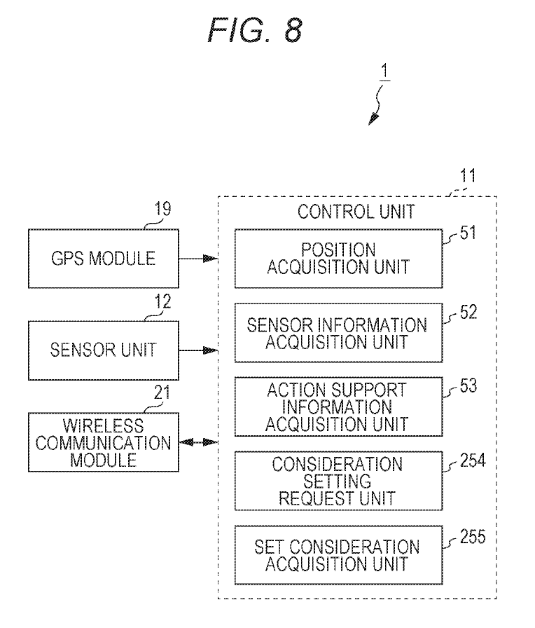

FIG. 8 is a functional block diagram indicating a functional configuration to perform terminal-side consideration setting processing among functional configurations of the wrist terminal 1 according to the second embodiment.

The terminal-side consideration setting processing is a series of processing to request setting of a consideration to the consideration setting server 100 and acquire information representing a consideration set from the consideration setting server 100 by acquiring user exercise information and user biological information by a sensor and sending the acquired exercise information or biological information to the consideration setting server 100.

In the case where the terminal-side consideration setting processing is performed, as illustrated in FIG. 8, a position acquisition unit 51, a sensor information acquisition unit 52, an action support information acquisition unit 53, a consideration setting request unit 254, and a set consideration acquisition unit 255 function in a control unit 11.

Among these, functional configurations of the position acquisition unit 51, the sensor information acquisition unit 52, and the action support information acquisition unit 53 are similar to a configuration of each unit illustrated in FIG. 2.

The consideration setting request unit 254 requests setting of a consideration for an action of a user of the wrist terminal 1 by sending, to the consideration setting server 100, position information of the wrist terminal 1 acquired by the position acquisition unit 51, user biological information and user exercise information acquired by the sensor information acquisition unit 52, and action support information acquired by the action support information acquisition unit 53. The action support information is sent to the consideration setting server 100 in the case where the information on user's action is acquired.

The set consideration acquisition unit 255 acquires information representing a consideration set by the consideration setting server 100 for setting of a consideration requested by the consideration setting request unit 254.

Next, a functional configuration of the consideration setting server 100 will be described.

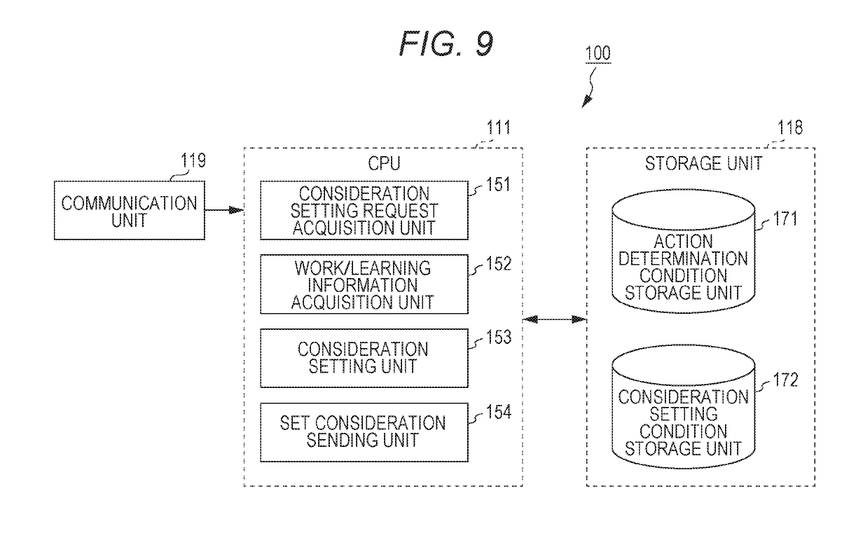

FIG. 9 is a functional block diagram indicating a functional configuration to perform server-side consideration setting processing among functional configurations of the consideration setting server 100 indicated in FIG. 7.

A server-side consideration setting processing is a series of processing to set information representing a consideration of a user's action based on position information of the wrist terminal 1, user biological information and user exercise information, and action support information in the case where the wrist terminal 1 requests setting of the consideration.

In the case where server-side consideration setting processing is performed, a consideration setting request acquisition unit 151, a work/learning information acquisition unit 152, a consideration setting unit 153, and a set consideration sending unit 154 function in the CPU 111, as illustrated in FIG. 9. Further, an action determination condition storage unit 171 and a consideration setting condition storage unit 172 are provided in a region of the storage unit 118.

Among these, configurations of an action determination condition storage unit 171 and a consideration setting condition storage unit 172 are similar to configurations of the action determination condition storage unit 71 and the consideration setting condition storage unit 72 illustrated in FIG. 2.

The consideration setting request acquisition unit 151 receives position information of the wrist terminal 1, user biological information and user exercise information, and action support information, sent from the wrist terminal 1.

The work/learning information acquisition unit 152 determines whether user's action pattern is associated with the work/learning information (in other words, acquires the work/learning information on user's action) with reference to an action determination condition table based on position information of the wrist terminal 1, user biological information and user exercise information, and action support information, sent from the wrist terminal 1. In the case where the work/learning information is not acquired, the work/learning information acquisition unit 152 sends, to the wrist terminal 1, information indicating that the work/learning information is not acquired. When receiving the information that the work/learning information is not acquired, the wrist terminal 1 notifies a user that the work/learning information is not acquired, for example, by displaying the information on the LCD 14. Then, after notifying the user that the work/learning information is not acquired, the wrist terminal 1 makes a user manually input the work/learning information, for example, via a touch panel. The consideration setting request unit 254 of the wrist terminal 1 accepts an input of the work/learning information by a user. A flag indicating manually input work/learning information is added to the acquired work/learning information by the consideration setting request unit 254. The work/learning information acquisition unit 152 receives, from the wrist terminal 1, the work/learning information in which the flag indicating manually input work/learning information is added.

With reference to a consideration setting condition table, the consideration setting unit 153 acquires “a consideration granting condition”, “a consideration granted amount”, “a consideration granting entity”, and “an authorization entity” (consideration setting conditions) based on the work/learning information determined that a user of the wrist terminal 1 has performed.

The consideration setting unit 153 calculates a consideration granted amount for the work/learning information determined that a user of the wrist terminal 1 has performed and specifies a consideration granting entity for an action of the user of the wrist terminal 1, based on position information of the wrist terminal 1 received by the consideration setting request acquisition unit 151, user biological information and user exercise information, and action support information. At this time, in the case where “an authorization entity” is set to the determined work/learning information, the consideration setting unit 153 requests authorization of the work/learning information to the authorization entity. In the case where the work/learning information is authorized by the authorization entity, the consideration setting unit 153 adds a consideration to a user of the wrist terminal 1. In this manner, the consideration setting unit 153 sets information representing a consideration for the work/learning information.

The set consideration sending unit 154 sends the information representing a consideration set by the consideration setting unit 153 to the wrist terminal 1.

As described above, based on position information of the wrist terminal 1, user biological information and user exercise information, and action support information, sent from the wrist terminal 1, the consideration setting server 100 acquires the work/learning information for an action of the user and sets information representing a consideration. As a result, authenticity of a consideration set to a user of the wrist terminal 1 can be enhanced.

Operation

Next, an operation of the consideration setting system S will be described.

First, an operation of the wrist terminal 1 is described.

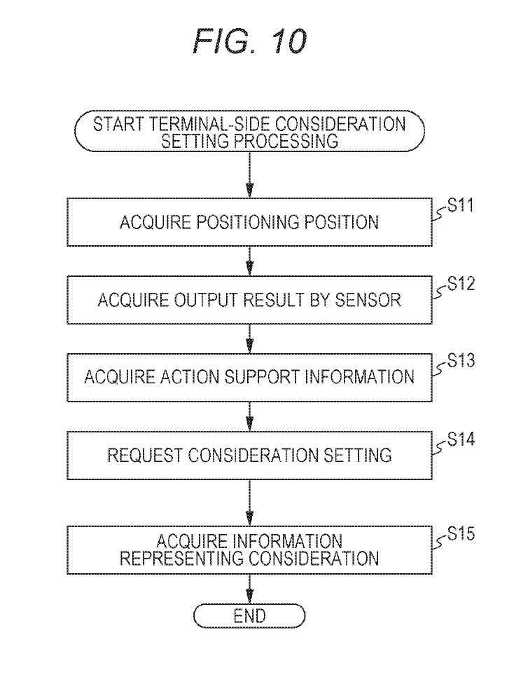

FIG. 10 is a flowchart describing a flow of terminal-side consideration setting processing performed by the wrist terminal 1 according to the embodiment.

The terminal-side consideration setting processing is started when a user operates the input unit 13 to start the processing.

In step S11, the position acquisition unit 51 acquires a positioning position of the wrist terminal 1 based on position information acquired from the GPS module 19.

In step S12, the sensor information acquisition unit 52 acquires output results of various sensors included in the sensor unit 12.

In step S13, the action support information acquisition unit 53 acquires action support information of a user of the wrist terminal 1 based on information output by the position acquisition unit 51 and the sensor information acquisition unit 52 and various user information stored in the wrist terminal 1.

In step S14, the consideration setting request unit 254 requests setting of a consideration for an action of a user of the wrist terminal 1 by sending, to the consideration setting server 100, position information of the wrist terminal 1 acquired by the position acquisition unit 51, user biological information and user exercise information user acquired by the sensor information acquisition unit 52, and action support information acquired by the action support information acquisition unit 53.

In step S15, the set consideration acquisition unit 255 acquires information representing a consideration set by the consideration setting server 100 for setting of a consideration requested by the consideration setting request unit 254.

After step S15, the terminal-side consideration setting processing is finished.

Next, an operation of a consideration setting server will be described.

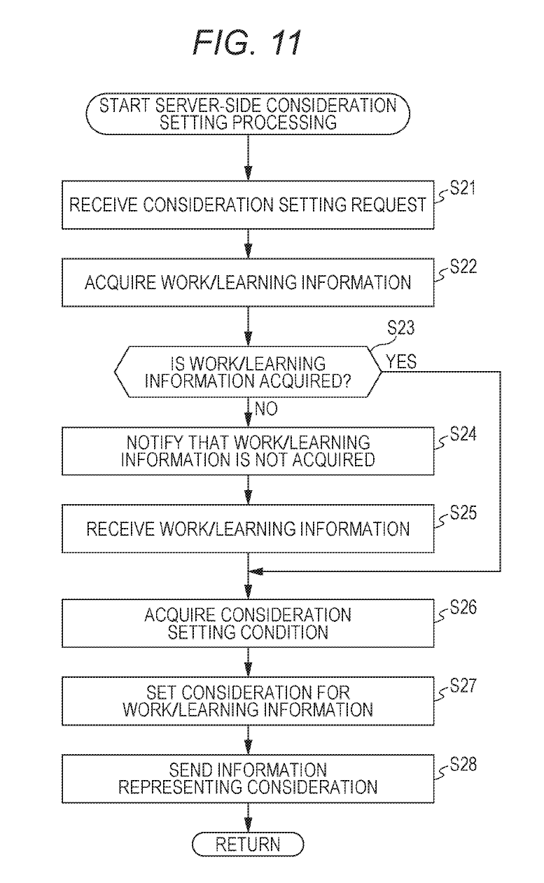

FIG. 11 is a flowchart describing a flow of the server-side consideration setting processing performed by the consideration setting server 100.

The server-side consideration setting processing is started when the consideration setting server 100 is started up, and the processing is repeatedly performed.

In step S21, the consideration setting request acquisition unit 151 receives, as a consideration setting request, position information of the wrist terminal 1, user biological information and user exercise information, and action support information, sent from the wrist terminal 1.

In step S22, the work/learning information acquisition unit 152 determines whether user's action pattern is associated with the work/learning information (in other words, acquires the work/learning information on user's action) with reference to an action determination condition table based on position information of the wrist terminal 1, user biological information and user exercise information, and action support information, sent from the wrist terminal 1.

In step S23, the work/learning information acquisition unit 152 determines whether the work/learning information is acquired.

In the case where the work/learning information is not acquired, it is determined NO in step S23, and step S24 is performed.

On the other hand, in the case where the work/learning information is acquired, it is determined YES in step S23, and step S26 is performed.

In step S24, the work/learning information acquisition unit 152 sends, to the wrist terminal 1, information indicating that work/learning information is not acquired.

In step S25, the work/learning information acquisition unit 152, as described above, receives, from the wrist terminal 1, the work/learning information in which a flag indicating manually input work/learning information is added.

In step S26, the consideration setting unit 153 acquires consideration setting conditions based on the work/learning information determined that a user of the wrist terminal 1 has performed, with reference to a consideration setting condition table.

In step S27, the consideration setting unit 153 sets information representing a consideration for the work/learning information.

In step S28, the set consideration sending unit 154 sends information representing a consideration set by the consideration setting unit 153 to the wrist terminal 1. In the case where a flag indicating manually input work/learning information is added to the work/learning information, a consideration for the work/learning information may be held while it is confirmed whether the work/learning information is normally performed. In this case, at a timing of step S28, a user may be notified that authenticity of the work/learning information is being confirmed. The authenticity of the work/learning information may be confirmed by directly contacting between a user and an authorization entity or a consideration granting entity.

After step S28, the server-side consideration setting processing is repeated.

As described above, the wrist terminal 1 according to the embodiment acquires user exercise information and user biological information by a sensor and sends the acquired user exercise information and user biological information to the consideration setting server 100. In the case where an action pattern of a user is determined to be associated with the work/learning information, the consideration setting server 100 sets information representing a consideration for the work/learning information associated with the action pattern and sends the information representing the set consideration to the wrist terminal 1.

Therefore, since a consideration for user's action is generated in accordance with a predetermined standard, the user can work and volunteer with high action motivation and high fairness and satisfaction.

Therefore, according to the wrist terminal 1, a value can be appropriately added to user's action, and the user's action can be further simply managed.

Further, the consideration setting server 100 acquires the work/learning information on user's action and sets information representing a consideration. As a result, authenticity of the consideration set to a user of the wrist terminal 1 can be enhanced.

First Variation

In the above-described embodiment, an RFID included in the wrist terminal 1 is read by an RFID reader disposed at an entrance of such as a facility, and the position acquisition unit 51 acquires information indicating a disposed position of the RFID reader and may set the disposed position to a position of the wrist terminal 1.

As a result, authenticity of data that a user of the wrist terminal 1 has been there can be secured.

For example, authenticity of data whether a user of the wrist terminal 1 receives a training for upskilling or whether the user takes a license course can be secured.

Second Variation

In the above described embodiments, the work/learning information acquisition units 54 and 152 may verify whether position information of the wrist terminal 1, user biological information and user exercise information, and action support information are appropriate. For example, regarding the position information of the wrist terminal 1, by verifying authenticity of a GPS signal which is the basis of the calculation, appropriateness of the position information can be verified. Further, regarding the user biological information and user exercise information, by performing pattern matching of data and a frequency analysis, it can be verified that the information is not artificially created. Further, regarding contents (such as a schedule) of the action support information, it can be verified with reference to objective information such as the internet that the information is not false.

As a result, the authenticity of a consideration set to a user of the wrist terminal 1 can be further enhanced, and the authenticity to add a value to user's action can be further enhanced.

Third Variation

In the second embodiment, in the case where setting a consideration is requested from the wrist terminal 1 to the consideration setting server 100, user biological information and user exercise information can be sent via a mediation server.

The mediation server verifies authenticity of data of such as personal authentication of a user, position information of the wrist terminal 1, user biological information and user exercise information and performs authorization to set information representing a consideration.

As a result, when a consideration for user's action is set, fraudulently setting the consideration can be certainly prevented.

Fourth Variation

In the above-described embodiments, a user using the wrist terminal 1 can be authorized by biological information such as a pulse.

As a result, it is prevented that a different user uses the wrist terminal 1 and requests to grant a consideration instead of an authorized user.

Fifth Variation

In the above-described embodiments, a distribution system for information representing a consideration set for user's action may be disposed.

In this case, by setting a type of information representing a consideration, a distribution can be prohibited, and predetermined conditions to allow the distribution can be set depending on the information type.

Sixth Variation

In the above-described embodiments, user′ action which is the basis of information representing a consideration may be recorded with a granted consideration.

In this case, for example, information representing a set consideration includes user's action which is the basis of the information.

As a result, it becomes possible to grasp information on such as an action on which information representing a consideration is based, and when a user uses information representing a consideration of a user, a third party can refer to this information.

Seventh Variation

In the above-described embodiment, a management (in other words, extinction and exchange) may be strictly performed in the case where information representing a consideration is used by generating the information as information which can be individually identified (for example, information including an identification number similar to a paper money) in addition that the information is used as a number.

As a result, counterfeiting and duplicating use of the information representing a consideration can be prevented, and the appropriate distribution environment for the information representing a consideration can be constructed.

As described above, the wrist terminal 1 includes the sensor information acquisition unit 52, a work/learning information acquisition unit 54, and a consideration setting unit 55.

The sensor information acquisition unit 52 acquires user exercise information and/or user biological information.

The work/learning information acquisition unit 54 acquires the work/learning information which is information representing a work or learning regarding a user based on the user exercise information and/or the user biological information.

The consideration setting unit 55 sets information representing a consideration for the work/learning information based on the work/learning information on the user, acquired by the work/learning information acquisition unit 54.

Therefore, since a consideration for user's action is generated in accordance with a predetermined standard, the user can act with high action motivation and high fairness and satisfaction.

Therefore, a value can be appropriately added to user's action, and the action can be further simply managed.

The wrist terminal 1 includes a ROM 16.

The ROM 16 accumulates user exercise information and/or user biological information acquired by the sensor information acquisition unit 52.

The work/learning information acquisition unit 54 acquires work/learning information based on the user exercise information and/or the user biological information accumulated by the ROM 16. As a result, the work/learning information associated with a series of user's motion can be acquired. In the case where this is applied in the second embodiment, exercise information and/or biological information accumulated in the ROM 16 of the wrist terminal 1 is transferred to the consideration setting server 100, and on the consideration setting server 100 side, the work/learning information may be acquired based on the exercise information and/or biological information accumulated in the wrist terminal 1.

The work/learning information acquisition unit 54 acquires work/learning information based on an action pattern which is an action unit in which user exercise information or user biological information acquired in a predetermined time period is compiled.

The consideration setting unit 55 sets information representing a consideration for the work/learning information based on the action pattern.

As a result, user's work/learning information can be acquired based on an action pattern.

The sensor information acquisition unit 52 acquires plural kinds of user exercise information and/or user biological information.

The work/learning information acquisition unit 54 acquires the work/learning information based on an action pattern including combination of plural kinds of the user exercise information and/or the user biological information.

The consideration setting unit 55 sets information representing a consideration for the work/learning information based on plural kinds of the user exercise information and/or the user biological information.

As a result, an action pattern including the combination of plural kinds of the exercise information or biological information is defined, and user's work/learning information can be acquired.

Further, the wrist terminal 1 includes the position acquisition unit 51.

The position acquisition unit 51 acquires position information of a user.

The work/learning information acquisition unit 54 acquires work/learning information of a user based on position information and exercise user information and/or user biological information.

As a result, with reference to position information of a user, work/learning information of the user can be acquired.

Further, the work/learning information acquisition unit 54 verifies whether user's exercise information and/or biological information acquired by the sensor information acquisition unit 52 is appropriate.

As a result, authenticity of information representing a consideration set to a user can be enhanced.

Further, the consideration setting unit 55 sets information representing a consideration for the work/learning information in the case where authentication of the work/learning information is requested to an authorization entity for the work/learning information, and the work/learning information is authorized.

As a result, information representing a consideration can be further appropriately set.

Further, the consideration setting system S includes the wrist terminal 1, the consideration setting server 100 (an embodiment of an electronic device according to the present invention).

The consideration setting server 100 includes the consideration setting request acquisition unit 151, the work/learning information acquisition unit 152, a consideration setting unit 153, and the set consideration sending unit 154.

The consideration setting request acquisition unit 151 acquires a request to set a consideration including user exercise information and/or user biological information from other electronic device.

The work/learning information acquisition unit 152 acquires work/learning information of a user based on user exercise information and/or user biological information.

The consideration setting unit 153 sets information representing a consideration for the work/learning information on a user based on the work/learning information on the user acquired by the work/learning information acquisition unit 152.

The set consideration sending unit 154 sends information representing a consideration set by the consideration setting unit 153 to the wrist terminal 1.

As a result, based on the user exercise information or user biological information acquired by the wrist terminal 1, a consideration can be set by the consideration setting server 100.

Therefore, while enhancing authenticity of a set consideration, a value can be further appropriately added to user's action.

The present invention is not limited to the above-described embodiments. Variations and improvements can be included as long as the object of the present invention can be achieved.

In the embodiments described above, user biological information and user exercise information are used when the work/learning information on user's action is acquired. However, biological information and exercise information may not be necessarily used. In other words, either of user biological information or user exercise information may be used when the work/learning information on user's action is acquired.

Further, in the above-described embodiment, as the work/learning information setting information representing a consideration, user's action in a game using such as augmented reality (AR) may be targeted.

Further, in the above-described embodiments, an example is described in which the wrist terminal 1 applied in the present invention is a wristwatch-type electronic device similar to a smart phone. However, the electronic device is not limited to the wristwatch-type in particular.

For example, the present invention can be applied to general electronic devices including an information processing function. More specifically, for example, the present invention can be applied to such as a note-type personal computer, a video camera, a portable-type navigation device, a cell phone, a smart phone, and a portable game machine.

A series of processing described above can be performed by hardware and software.

In other words, functional configurations indicated in FIGS. 2, 8, and 9 are indicated for an example and not particularly limited. Specifically, as long as the wrist terminal 1 or the consideration setting server 100 has a function which can perform the above-described series of processing as a whole, a function block using to realize the function is not limited to an example indicated in FIG. 2 in particular.

Further, one function block may include either of hardware or software and may include combination of hardware and software.

A functional configuration in the embodiment is realized by a processor performing arithmetic processing. The processor capable of being used in the embodiment includes various processing devices, such as a single processor, a multiprocessor, and a multi-core processor. Further the processor may include combination of these processing devices and a processing circuit, such as an application specific integrated circuit (ASIC) and a field-programmable gate array (FPGA).

In the case where a series of the processing is performed by software, a program included in the software is installed from the network and a storage medium in such as a computer.

The computer may be a computer incorporated into a special hardware. Further, the computer may be a computer, for example, a general-purpose personal computer, capable of performing various functions by installing each program.

A storage medium including such a program includes a storage medium provided to a user in a state in which the storage medium is incorporated into a device in advance, in addition to including the removable media 31 illustrated in FIG. 1 distributed different from a device to provide a program to a user. The removable media 31, for example, includes a magnetic disk (including a floppy disk), an optical disk, or an optical magnetic disk. The optical disk includes, for example, a compact disk-read only memory (CD-ROM), a digital versatile disk (DVD), a Blu-ray (registered trade mark) Disc (Blu-ray disk). The optical magnetic disk includes such as a Mini-Disk (MD). The storage medium provided to a user in a state of being stored in a device includes, for example, a hard disk included in the ROM 16 illustrated in FIG. 1, the ROM 112 illustrated in FIG. 7, and the storage unit 118 illustrated in FIG. 7, in which a program is recorded.

In the description, a step describing a program stored in a storage medium includes processing time-serially performed in turn and also parallely or individually performed even if the processing is not time-serially performed.

Further, in the description, system terms mean an overall device including a plurality of devices and a plurality of units.

As described above, embodiments of the present invention have been described above. However, these embodiments are indicated as an example and not for limiting a technical range in the present invention. The present invention can apply other various embodiments and can be variously changed such as abbreviation and replacement without departing from the scope of the present invention. These embodiments and variations are included in the range and scope of the invention described in the description and included in the range equivalent to the invention described in claims.

What is claimed is:

1. An electronic device comprising a processor configured to
acquire user exercise information and/or user biological information,
acquire work information representing a work regarding a user or learning information representing learning regarding the user based on the user exercise information and/or the user biological information, and
set information representing a consideration based on the work information or the learning information.

2. The electronic device according to claim 1, wherein the processor is further configured to
accumulate the user exercise information and/or the user biological information, and
acquire the work information or the learning information based on the accumulated user exercise information and/or user biological information.

3. The electronic device according to claim 1, wherein the processor is further configured to
acquire the work information or the learning information based on an action pattern which is an action unit in which the user exercise information or the user biological information is compiled, which is acquired in a predetermined time period, is combined, and
set information representing the consideration based on the action pattern.

4. The electronic device according to claim 3, wherein the processor is further configured to
acquire plural kinds of the user exercise information and/or plural kinds of the user biological information,
acquire the work information or the learning information based on the action pattern combining the plural kinds of user exercise information and/or the plural kinds of user biological information, and
set information representing the consideration based on the plural kinds of user exercise information and/or the plural kinds of user biological information.

5. The electronic device according to claim 1, wherein the processor is further configured to
acquire position information of the user, and
acquire the work information or the learning information based on (i) the position information and (ii) user exercise information and/or user biological information.

6. The electronic device according to claim 1, wherein the processor is further configured to verify whether the acquired user exercise information and/or user biological information is appropriate.

7. The electronic device according to claim 1, wherein the processor is further configured to request authentication of the work information or the learning information to an entity for authenticating the work information or the learning information, and set information representing the consideration in a case where the work information or the learning information is authenticated.

8. The electronic device according to claim 1, wherein the processor is further configured to
acquire the user exercise information and/or the user biological information, which is detected by an external device, and
send the set information representing the consideration to the external device.

9. A consideration setting method performed by an electronic device comprising a processor, configured to acquire user exercise information and/or user biological information, the consideration setting method comprising the steps of:
acquiring work information representing a work regarding a user or learning information representing learning regarding the user based on the user exercise information and/or the user biological information; and
setting information representing a consideration based on the acquired work information or the acquired learning information.

10. A non-transitory computer-readable medium comprising a program stored thereon, which, when executed on a processor, realizes:
an exercise/biological information acquisition function configured to acquire user exercise information and/or user biological information;
a work/learning information acquisition function configured to acquire work information representing a work regarding a user or learning information representing learning regarding the user based on the user exercise information and/or the user biological information; and
a consideration setting function configured to set information representing a consideration based on the work information or the learning information acquired by the work/learning information acquisition function.
